(19) United States
(12) Patent Application Publication
(10) Pub. No.: US 2007/0298024 A1
(43) Pub. Date: Dec. 27, 2007

(54) C-1 inactivator inhibits two-chain urokinase mutant and limits hemostatic bleeding during thrombolysis

(75) Inventor: Gurewich Victor, Cambridge, MA (US)

Correspondence Address: Kevin M. Farrell;Pierce Atwood, Suite 350, One New Hampshire Avenue, Portsmouth, NH, 03801, US

(73) Assignee: Thrombolytic Science, Inc., Cambridge, MA (US)

(21) Appl. No.: 11/472,607

(22) Filed: Jun. 22, 2006

(51) Int. Cl. A61K 38/48

(52) U.S. Cl. 424/94.64

(57) ABSTRACT

Methods for reducing bleeding during fibrinolysis treatment and for inhibiting the enzymatic activity of a two-chain urokinase mutant are described. Exogenous C1-inactivator is administered during fibrinolysis treatment with the pro-urokinase mutant polypeptide, M5. The C1-inactivator inhibits the formation of two-chain M5 resulting in less hemostatic bleeding.

### BACKGROUND OF THE INVENTION

Existing thrombolytic drugs, used in the treatment of thromboembolic diseases, have limited effectiveness and also carry the risk of rethrombosis and hemorrhagic complications. Currently most therapeutic thrombolysis is performed using the single chain enzyme, tissue plasminogen activator (tPA), and its derivatives. TPA can have hemorrhagic side effects. Single-chain urokinase or prourokinase (proUK) is a natural, alternative plasminogen activator. Both tPA and proUK are fibrin-specific in that they prefer to lyse plasminogen that is bound to fiber over free plasminogen.

In clinical trials, proUK was discovered to be subject to spontaneous activation in plasma. This resulted in non-specific fibrinolysis mediated by UK (Meyer et al., Lancet 1989: 1863-1868). The intrinsic activity of proUK at therapeutic concentrations was sufficient to activate plasma plasminogen, which converted proUK to UK. Whether or not proUK is fibrin-specific depends on its plasma stability allowing UK and plasmin generation to be confined to the fibrin clot (Pannell and Gurewich, Blood, 67: 1215-1223 (1986)). The activation of plasma plasminogen results in the generation of systemic UK and undermines the therapeutic use of proUK.

Improving the plasma stability of proUK will make proUK therapeutically effective. The structural basis of proUK's high intrinsic activity was investigated. A flexible loop in the catalytic domain which contained a critical positively charged residue was identified (Liu et. al., Biochemistry 35: 14070-14076 (1996)). M5 is a single site, Lys300→His proUK mutant which has been characterized as having a reduced charge at this site. The intrinsic activity of M5 is one-fifth that of proUK. The intrinsic activity of two-chain M5 (tcM5) is almost twice that of UK (Sun et al., J Biol Chem. 272: 23818-23823 (1997)). U.S. Pat. No. 5,472,692 describes proUK mutants and the disclosure is incorporated herein by reference.

Clot lysis in a plasma environment with M5 was compared to that with proUK. The fibrin-specificity of M5 was retained over a dose range five times as wide as that of proUK. Clot lysis was also faster with M5, probably due to the mutant's higher two-chain activity. Comparable results were obtained in in vivo experiments in dogs. When autologous radiolabeled blood clots were embolized to dogs' lungs, the bleeding time and blood loss from a standardized incision was lower with M5 than with either pro-UK or tPA suggesting that M5 spares hemostatic fibrin at doses which lyse intravascular clots (Liu et al., Circ Res. 90: 757-763 (2002)).

In a second study, a more challenging arterial thrombus was selected and M5 was administered with a bolus/infusion modeled on the clinical administration of proUK and tPA. Because blood loss from injury sites was the side effect of most concern, a more quantitative measure of blood loss was used. Furthermore, the plasma inhibition of the non-specific tcM5 effect was studied and found to be related to a plasma inhibitor novel for UK.

### SUMMARY OF INVENTION

A single site mutant (M5) of prourokinase (proUK) was developed to make proUK less subject to spontaneous activation in plasma during fibrinolysis. The spontaneous activation precluded proUK-mediated fibrinolysis at therapeutic concentrations and seriously compromised proUK in clinical trials.

After dose-finding studies were completed, twelve dogs with femoral artery thrombosis were anesthetized and given either M5 (2.0 mg/kg) or tPA (1.4 mg/kg) by intravenous infusion over 60 minutes (20% administered as a bolus). Two pairs of standardized injuries were inflicted on each dog and hemostasis was completed followed by drug administration. Blood loss was quantified by measuring the hemoglobin in blood absorbed from these injury sites. Thrombolysis was evaluated at 90 minutes and was comparably effective by both activators. Rethrombosis developed in one tPA dog. Blood loss was ten times higher with tPA (mean˜40 ml) than with M5 (mean ˜4 ml) (p=0.026) and occurred at more sites per dog (mean 2.7 vs 1.2). It was postulated that this effect was due to differences in the mechanism of plasminogen activation by tPA and M5. M5 is promoted by degraded rather than intact (hemostatic) fibrin. A second circumstance contributing to the reduced blood loss in the M5 treated dogs is that tcM5 is efficiently inactivated by plasma C1-inactivator, an exceptional property which helps contain its non-specific proteolytic effect.

Intravascular thrombolysis by M5 was accompanied by significantly less bleeding from hemostatic sites than was thrombolysis by tPA. When the M5 mutation was used fibrinolysis was kept at therapeutic levels.

### BRIEF DESCRIPTION OF DRAWINGS

FIG. 1. Radioisotope counts per minute (mean and one STD) as a percent of the initial count registered by a gamma probe stationed over the femoral artery segment containing the clot formed in the presence of ¹²⁵I-labeled fibrinogen.

FIG. 2A. Relative sizes of the residual thrombi found in the femoral artery segments when they were opened at 90 minutes. Size graded 1-4+, 1+ representing a small fleck and 4+ a cast of the vessel.

FIG. 2B. Digital photos of the thrombi (arrows) found, along with the excised vessel segments. The two M5 segments with residual thrombi are on the left and the three tPA are on the right. The 4+ thrombus seen in the first of the three tPA segments was non-adherent and emitted little radioactivity and was probably related to rethrombosis.

FIG. 3. Number of sites in each dog from which significant (>1,000 ODU or >1.3 ml) bleeding occurred. Blood loss was calculated from a measurement of the hemoglobin shed at each of the 4 sites.

FIG. 4. Total blood loss (sum of the 4 wound sites) for M5 (mean˜4 ml) and for tPA (mean˜40 ml). 75,000 ODU was equivalent to ˜100 ml whole blood.

FIG. 5. Representative zymograms of plasma in which either tcM5 or UK (5 μg/ml) were incubated. MW marker kDa's are shown in the first lane and minutes across the top. Both tcM5 and UK appear as higher (˜45 kDa) and lower (˜30 kDa) MW lysis zones. The prominent ˜150 kDa inhibitor complex (consistent with C1-inactivator) was seen within minutes with tcM5, whereas it was delayed and only faintly visible with UK. The second inhibitor complex at ˜110 kDa (consistent with antithrombin) was also more prominent with tcM5 than UK. As shown, there was a correspondingly more rapid loss of free tcM5 activity compared with UK.

FIG. 6. Studies of tcM5:C1-inactivator complexes in plasma (A) and in mixtures of purified tcM5 and C1-inactivator (B). A: Zymogram of tcM5 in plasma showing the complex. The lower fainter complex probably represents antithrombin (1). Western blot of this plasma showing the complex with UK antibodies (2). Western of tcM5 (3). Western of the plasma showing the complex with C1-inactivator antibodies (5). B: Zymogram of tcM5 (1). Zymogram of mixture of tcM5 and C1-inactivator showing complexation (3). Coomassie SDS PAGE of tcM5 (4). Coomassie of tcM5 plus C1-inactivator showing the complex (5). Coomassie of C1-inactivator (6). MW markers (A2, B2, 7).

### DETAILED DESCRIPTION OF THE INVENTION

The present invention relates to a novel method of reducing bleeding during fibrinolysis treatment. The method is based on the discovery that C1-Inactivator has the ability to inhibit the formation of the two-chain prourokinase mutant tcM5. Prourokinase (ProUK) is a thrombolytic drug with the undesirable side effect of being vulnerable to spontaneous activation in plasma during fibrinolysis. M5 is a single site mutant of prourokinase developed to limit fibrinolysis to a local target area and to reduce hemostatic fibrinolysis. M5 differs from prourokinase by a single amino acid substitution at position 300, where the amino acid Lysine has been replaced by Histidine. C1-inactivator is a previously unknown plasma inhibitor of UK. C1-inactivator is a serine protease inhibitor normally present in blood at levels ranging from 0.25-0.45 g/l. Deficiency and dysfunction of this protein have been associated with diseases such as hereditary angioedema.

As discussed in the Background of the Invention section, ProUK is a plasminogen activator that is subject to spontaneous activation in plasma. Plasminogen converts proUK into UK and plasmin, resulting in non-specific fibrinolysis by UK, wherein UK and plasmin generation are not confined to the target fibrin clot. M5 has only one-fifth the intrinsic activation of proUK. When used to lyse intravascular blood clots, M5 largely spares hemostatic fibrin. For the present invention, experiments were performed in vivo in dogs and in vitro in human and dog plasma. The actions of M5 were studied in comparison to tissue plasminogen activator (tPA), currently the most commonly used thrombolytic drug.

tPA and proUK are both natural plasminogen activators, and both induce fibrin-specific lysis by preferentially activating fibrin-bound plasminogen over free plasminogen. However, distinctly different mechanisms are responsible for this phenomenon. Each activator targets a different fibrin-bound plasminogen. tPA is a single-chain enzyme with a high affinity for a specific binding site on fibrin, where tPA forms a ternary complex with an adjacent plasminogen (Hoylaerts et al., J Biol Chem. 257: 2912-2919 (1982)). This plasminogen is bound to an internal lysine binding site (Lys-157) in the Aα chain of the D-region of fibrin (Nieuwenhuizen W et al., Biochim Biophys Acta. 748: 86-92 (1983)). In the presence of fibrin fragment D, plasminogen activation by tPA is promoted by as much as 1,000-fold (Petersen et al., Biochim Biophys Acta. 952: 245-254 (1988)), reflecting the importance of the ternary complex for tPA.

By contrast, the single-chain proUK has no fibrin affinity. Yet when a clot is added to plasma containing proUK (or MS), local activation of a fraction of the proUK takes place on the fibrin surface and lysis is triggered (Liu et. al., Biochemistry 35: 14070-14076 (1996)). This sequence of events is facilitated by a conformational change in plasminogen for which proUK (or M5) has high substrate affinity. This change occurs when plasminogen binds to its carboxy-terminal lysine binding site in the E region of fibrin. In the presence of fibrin fragment E, plasminogen activation by single-chain proUK/M5 is equal to that of its two-chain derivative, UK or tcM5, corresponding to a several hundred-fold promotion of its intrinsic activity (Liu and Gurewich, Biochemistry 31: 6311-6317 (1992)).

Therefore, tPA and proUK (M5) both induce fibrin dependent plasminogen activation, dependent on an internal lysine in the fibrin D region for tPA and dependent on carboxy-terminal lysines in the E region for proUK (M5). This difference is selective since there is little or no reciprocity. In a purified system, plasminogen activation by tPA is promoted specifically by fibrin fragment D and that by proUK/M5 is promoted only by fibrin fragment E (Liu and Gurewich, J Clin Invest. 88: 2012-2017 (1991)). Newly formed intact fibrin contains only the internal lysine plasminogen binding site in the D region of fibrin. The carboxy-terminal lysines in the E region are created only after plasmin degradation has occurred (Harpel et al., J Biol Chem. 260: 4432-4440 (1985)). This is evidenced by the lag phase of proUK-induced clot lysis in a plasma milieu and by the fact that the lag phase is substantially attenuated by gentle pre-treatment of the clot with plasmin. Conversely, tPA lyses intact and degraded clots equally well under these same conditions (Pannell et al., J Clin Invest. 81: 853-859 (1988)). Thus, intact fibrin is relatively resistant to lysis by proUK/M5 but not by tPA.

Hemostatic fibrin, consistent with its physiological function, is protected from plasmin degradation by several physiological safeguards. These include the inhibition of free tPA (and UK) at physiological levels by plasminogen activator inhibitor type-1 and the removal of carboxy-terminal binding sites on fibrin by thrombin-activated procarboxypeptidase in plasma (Hendriks et al., J Clin Chem. Clin Biochem. 27: 277-280 (1989)). It is consistent with the present findings that hemostatic fibrin should correspond to intact fibrin. By contrast to hemostatic fibrin, when an intravascular thrombus forms and causes a vascular occlusion, physiological mechanisms for its dissolution are triggered. In particular, there is a release of tPA from the vessel wall which, aided by the local stasis, binds to the thrombus. Fibrin degradation is initiated thus creating new carboxy-terminal lysine plasminogen binding sites which facilitate lysis (Harpel et al., J Biol Chem. 260: 4432-4440 (1985)), particularly by proUK/M5.

The presence of the fibrin E region plasminogen binding sites in an intravascular thrombus but not in hemostatic fibrin provides an explanation for why effective thrombolysis by M5 spares hemostatic fibrin. In contrast, the D region ternary complex plasminogen binding site is present in hemostatic fibrin. Its presence is consistent with tPA-associated bleeding and provides an explanation for its low correlation with fibrinogen degradation (Montoney et al., Circulation 91: 1540-1544 (1995)), being more related to direct lysis of hemostatic (intact) fibrin than to a non-specific effect.

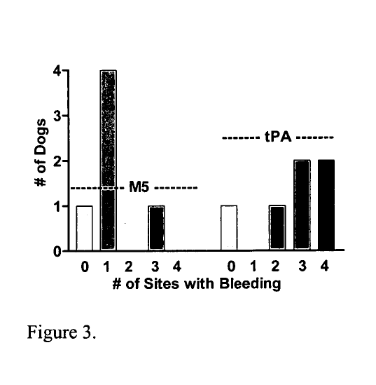

As discussed in the Exemplification section, rebleeding from hemostatic sites was measured during treatment with either M5 or tPA. Dogs were inflicted with standardized injuries and hemostasis was completed before administration of tPA or M5. The efficacy of thrombolysis by M5 and tPA was comparable, but intravascular thrombolysis by M5 was accompanied by significantly less bleeding from hemostatic sites than was thrombolysis by tPA. Total blood loss was ten times higher in dogs treated with tPA (mean˜40 ml) than with M5 (mean˜4 ml). With tPA, blood loss also occurred at more sites per dog (mean 2.7 vs 1.2, see FIG. 3), making it unlikely to be related to local variables such as inter-animal differences in the incisions. M5 caused effective lysis of lung clots in dogs with little bleeding from two hemostatic sites. The findings indicate a relative sparing of hemostatic versus intravascular fibrin when M5 was used and suggest that these two fibrins are functionally distinct with respect to their sensitivity to lysis by M5.

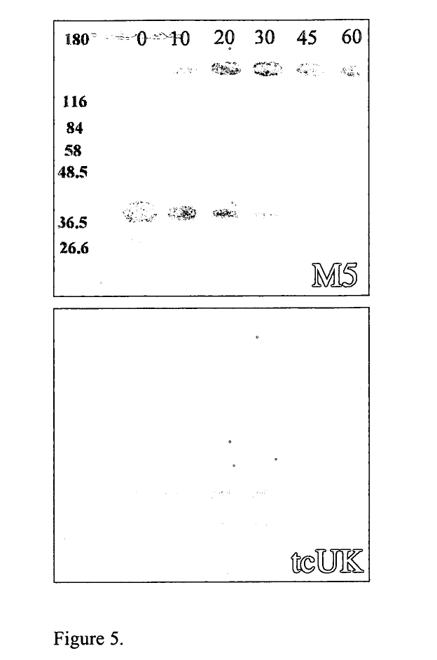

When the enzymes tcM5 and UK were each incubated in vitro in either human or dog plasmas, an inhibitor complex consistent with C1-inactivator (˜150 kDa) appeared within minutes of the incubation of tcM5 in plasma. These findings were reproducible and comparable in human and dog plasma. A representative zymogram from an experiment in human plasma is shown (FIG. 5).

Zymograms from plasma samples obtained in vivo from the M5-treated dogs showed only a ˜45 kDa band with no detectable complexes, indicating little systemic tcM5 generation (consistent with the little fibrinogen degradation).

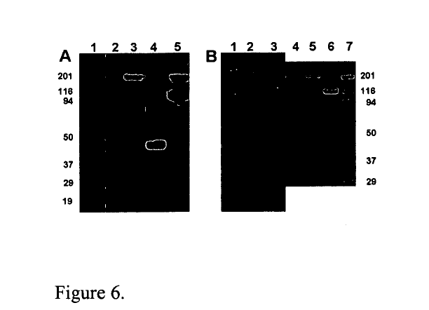

Western blotting (FIG. 6) with antibodies to UK and to C1-inactivator was performed on plasma samples containing tcM5. The blots revealed complexes migrating in the same position relative to each other and in the same position as that of the predominant complex in a zymogram of the plasma. Studies in a purified system with C1-inactivator and tcM5 confirmed that a complex formed between tcM5 and C1-inactivator and migrated to the same position as that seen on the plasma zymograms. Coomassie staining of SDS PAGE of tcM5, tcM5 plus C1-inactivator, and C1-inactivator showed the same enzyme:inhibitor complex.

The efficient inhibition of tcM5 by C1-inactivator also contributed to protecting hemostatic fibrin. During thrombolysis by M5, tcM5 is generated, which like UK, is a non-specific plasminogen activator. Only plasma inhibitors can confine tcM5 activity to the clot environment. As shown by the zymograms of plasma in which tcM5 or UK were incubated (FIG. 5), tcM5 was more efficiently inhibited, mostly due to complexation with C1-inactivator (FIG. 6). C1-inactivator has not previously been included as a UK inhibitor (Murano, et al., Blood 55: 430-436 (1980)), though it is a weak tPA inhibitor (Huisman, Thromb Haemost. 73: 466-471 (1995)). Only a very faint C1-inactivator complex with UK was seen in the present study (FIG. 5). Although these complexes were not detectable in the plasma sample zymograms obtained from the M5 dogs in the study, the inhibition of tcM5 in plasma is what limits the chain reaction which would otherwise result in more tcM5 formation, plasmin generation, and bleeding. At higher M5 doses which were used during dose-finding, inhibitor complexes comparable to those in vitro were seen in the dogs.

These results make clear that administering an amount of exogenous C1-inactivator along with M5 during fibrinolysis treatment would limit non-specific plasminogen activation in the blood and reduce bleeding. An amount of M5 effective to cause lysis of an occlusive blood clot should be administered along with an amount of exogenous C1-inactivator sufficient to limit non-specific plasminogen activation in blood. The M5 mutant is administered as a thrombolytic agent in the same way as pro-UK and UK. M5 is mixed with a pharmaceutically acceptable carrier, e.g., saline, and administered by intravascular, e.g., intravenous or intra-arterial, or subcutaneous injection. M5 is injected as a bolus of approximately 20 to 60 mg, or may be infused intravenously at a rate of 40-80 mg/hour. Since M5 has far greater plasma stability than native pro-UK, and is less likely to induce non-specific plasminogen activation, higher dosages, e.g., infusions of up to 200 mg/hour may also be used.

The present invention includes the treatment of a patient with M5 and an amount of C1-inactivator sufficient to establish C1-inactivator concentrations substantially greater than physiological levels in the plasma of the patient. The concentrations established should be about two to three times that of physiological levels or approximately 0.75 g/l to 1.5 g/l.

Another embodiment of the invention is a method of inhibiting the enzymatic activity of tcM5. As is detailed in the Exemplification, an amount of exogenous C1-inactivator sufficient to limit the formation of tcM5 from M5 activation should be administered to a patient desiring to limit the non-specific activity of tcM5.

M5 and tPA induced lysis of a femoral artery thrombus in dogs with comparable efficacies, but M5 caused ten-fold less (p=0.026) bleeding from wound sites due to an apparent sparing of hemostatic fibrin. This was postulated to be due to the finding that plasminogen activation by M5 was not promoted by intact (hemostatic) fibrin, and to the efficient inhibition of tcM5 by plasma C1-inactivator.

### EXEMPLIFICATION

### Materials

Recombinant Lys300→His proUK expressed in Escherichia coli was prepared as previously described (Liu et al., Circ Res. 90: 757-763 (2002)) and obtained from Primm (Milan, Italy). Single-chain tPA, pharmaceutical grade, was purchased from Genentech (San Francisco, Calif.). Recombinant proUK expressed in E. coli was obtained from Landing Science and Technology Company, Nanjing, China. Aprotinin was obtained as Trasylol from Miles, Inc., Kankakee, Ill. Purified human C1-inactivator was obtained from ZLB Behring, Germany.

### Methods

Fibrinogen was measured as thrombin clottable protein. Plasma (0.5 ml) was diluted with 2 volumes of 0.06 M sodium phosphate, pH 6.1. One volume of thrombin (100 NIH units/ml; ThromboMax from Sigma, St. Louis, Mo.) was added and mixed and incubated for 30 min at 37° C. The clot was wound onto a wooden stick to express the diluted serum proteins, rinsed by standing in 5 ml of the buffer; then deposited into a tube with 1 ml of 5% NaOH. After boiling for 1 min, the clot was dissolved and the protein was measured spectrophotometrically at 280 nm.

Laemmli SDS-PAGE electrophoresis was carried out in 10% polyacrylamide slab gels. Zymography was performed according to the method of Granelli-Piperno and Reich (Granelli-Piperno and Reich, J Exp Med. 148: 223-234 (1978)) as modified by Vassalli et al (Vassalli et al., J Exp Med. 159: 1653-1658 (1984)). After electrophoresis, the polyacrylamide slabs were washed by agitation for 2 hours in 2.5% Triton X-100 in water, followed by 1 hour in 0.1 M Tris-HCl (pH 8.0), and then layered over an underlay consisting of 0.8% agarose (Agarose low melting, Fisher Biotech), casein (2% w/v; Carnation Non-fat Dry Milk), and plasminogen (20 μg/ml) in 0.1 M Tris-HCl (pH 8.0). With incubation the electrophoretic bands of plasminogen activator produced a cleared zone in the white casein background. Inhibitor complexes become active in this system.

For western blotting, proteins were transferred to nitrocellulose membrane (Amersham Biosciences) and probed with specific antibodies to urokinase (American Diagnostica) and to C1-inactivator (ZLB Behring, Germany) and developed with the Pierce Supersignal West Dura Kit.

### In Vivo Studies

All procedures in animals were in accordance with the Guide for the Care of Animals (National Academy of Science, 1996) and were approved by the Animal Studies Committee at the University of Pittsburgh, McGowan Institute of Regenerative Medicine.

### Thrombolysis Animal Model

Dogs were chosen as the experimental animal for these studies because of the well-established species specificity of proUK/UK. Dogs are one of the few animals comparably sensitive to the human enzyme as man. The animal model of arterial thrombosis described by Badylak et al. (J Pharmacol Methods 19: 293-304 (1988)) and previously used to evaluate proUK (Badylak et al., Thromb Res. 52: 295-312 (1988)) was used for M5 in this study. All the animal experiments were performed at the University of Pittsburgh. In brief, female beagle dogs weighing 7-10 kg were anesthetized with pentobarbital and maintained at a surgical plane of anesthesia with isoflurane. The left femoral artery, with the associated profunda branch, was isolated. The profunda femoris branch was cannulated (PE 0.5 mm ID) to provide access to the segment. Proximal and distal ligatures were placed in order to delineate a 1.5-2 cm segment of the vessel. After extracting all blood from the segment using a syringe, the segment was filled with hot (>90° C.) saline for 5 minutes. After a 5-minute exposure, the saline was removed, and blood flow restored through the segment for 20 seconds, following which the segment was allowed to fill with blood by retightening the distal ligature followed by the proximal. A tracer of 15 μCi of ¹²⁵I-labelled fibrinogen (Amersham Corp., Arlington Heights, Ill.) was then instilled into the segment through the access branch and thoroughly mixed, followed by 100 units of thrombin (Sigma, St. Louis, Mo.) in 0.05 ml saline. At the end of 15 minutes, the proximal ligature was opened allowing some contact with the circulation, and at the end of 30 minutes, the time needed for the clot to become fully adherent to the vessel wall, the distal ligature was opened in preparation for the infusions.

The radioactivity over the thrombus was monitored continuously for 90 minutes with a ¹²⁵I-specific gamma probe (Eberline Co., Santa Fe, N.Mex.) positioned over the femoral artery segment. After 90 minutes, the segment was isolated by double ligatures at each end, removed by cutting between the ligatures, and its contents examined after opening and spreading the vessel. Residual clot was graded 1-4+ with 1+ representing one or two small flecks and 4+ a larger clot filling the segment. The open vessel and its contents were photographed with a digital camera.

### Infusion of M5 or tPA.

Dose finding experiments in this model were first undertaken in order to establish the dose of each activator which was both effective and relatively fibrin-specific, defined as fibrinogen consumption of ≦40%. Doses of 2 mg/kg M5 and 1.4 mg/kg tPA were selected on that basis, of which 20% was administered as a bolus by push with a 10 ml syringe and the remainder by constant infusion over 60 minutes with an infusion pump (Harvard). Compared with the previous study (Liu et al., Circ Res. 90: 757-763 (2002)), the dose of M5 in the present study was about 40% less, whereas the tPA dose was 40% more. Each activator was made up in a solution containing 1 mg/ml and administered via a catheter in the jugular vein of the dog. Twelve dogs were infused alternatively with M5 or tPA.

Significant endogenous lysis in this model does not occur in 90 minutes (Badylak et al., J Pharmacol Methods 19: 293-304 (1988), Badylak et al., Thromb Res. 52: 295-312 (1988)). Therefore, a placebo group was not included.

### Experimental Model of Rebleeding.

In each animal, two pairs of previously standardized injuries were made and evaluated during the dose-finding stage of this study. Over the right and left sides of the shaved upper abdomen of the anesthetized dog, a 1 cm² skin-deep incision was made from which the epidermis was peeled off as previously described (Liu et al., Circ Res. 90: 757-763 (2002)). One of the exposed small vessels in the dermis at each site was cut until bleeding ensued and then dabbed at intervals until hemostasis.

Secondly, after removing the hair from the dorsal surface of each ear, a full thickness incision was made 3-5 mm in length using a #11 scalpel blade in an area devoid of visible vessels, as previously described in rabbits (Marder et al., Thromb Res. 67: 31-40 (1992)). The bleeding points on both surfaces of each ear were dabbed at intervals until hemostasis.

Administration of M5 or tPA was not started until bleeding at all four sites had ceased. When rebleeding occurred during the infusions, the blood was absorbed into gauze pads over the 90-minute duration of the experiment. The gauze pads from each of the bleeding sites were collected separately into plastic bags and analyzed the following day as follows: The gauze pads was placed in a measured amount of distilled water to hemolyze the red cells allowing the hemoglobin to go into solution (the gauze returned to its original white in the water). The hemoglobin concentration was then measured by spectrophotometry at 410 nm (in optical density units, ODU). For a hematocrit of 40-45%, which was average for these dogs, 75,000 ODU corresponded to about 100 ml whole blood.

### Blood Sampling.

Blood samples were collected from the jugular vein contralateral to the one used for the infusion. Samples were collected into tubes containing citrate (1:9) and aprotinin (200 KIU/ml final concentration) and were obtained at baseline, 55 minutes and 90 minutes.

Fibrinogen concentrations were measured in all samples and expressed as a percent of the baseline value. The 55-minute sample was also used for zymography to evaluate inhibitor complexes and to estimate the M5 concentration.

### Incubation of tcM5 and UK in Plasma.

The stability of M5 or proUK in plasma depends on the efficiency by which tcM5 or UK is inhibited. Without inhibitors, a mixture of M5/proUK and plasminogen will spontaneously convert to tcM5/UK and plasmin, though this does occur less rapidly with M5 than with proUK. Therefore, the efficiency of inhibition of tcM5 by plasma inhibitors is relevant and was evaluated in comparison with UK in dog and human citrate plasma. M5 and recombinant proUK from E. coli were each activated with plasmin by the method of Pannell and Gurewich (Pannell and Gurewich, Blood 1987;69: 22-26 (1987)). The kinetics of plasmin activation of M5 and proUK are comparable. Each enzyme (5 μg/ml) was then incubated (37° C.) in citrate plasma (human or dog) and samples removed for zymography at time intervals for 60 minutes. The experiments were repeated several times using different sampling intervals. The in vitro zymograms were compared with zymograms obtained from the 55-minute samples from certain dogs in the M5 dose-finding study in which inhibitor complexes were detectable (higher doses).

### Statistics

Statistical analysis was by the Mann Whitney two-tailed test using GraphPad Prism version 3.03 for Windows, GraphPad Software, San Diego, Calif. USA.

### Results

### Lysis of the Femoral Artery Thrombus

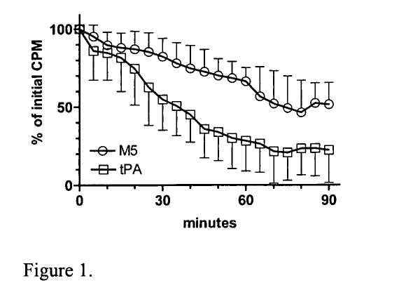

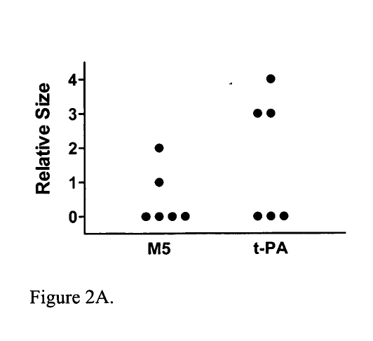

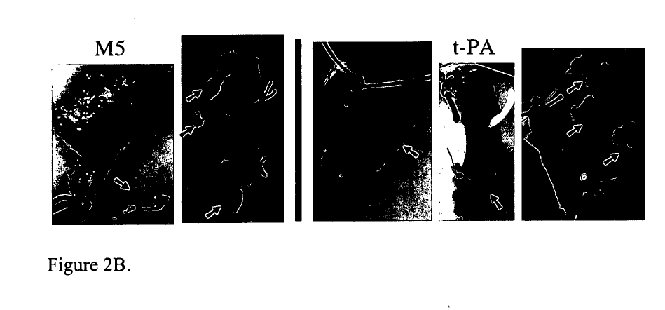

A gradual decline in radioactivity recorded over the segment occurred during the infusions with each activator. After 90 minutes, the radioactivity reached about 20% of baseline with tPA and about 50% with M5 (FIG. 1), suggesting superior lysis by tPA. However, this was found not to be the case when the segments were opened and examined. In 3 of the tPA dogs, large clots (3-4+) were found, of which one was due to rethrombosis (see below). In the M5 dogs, 2 residual thrombi were found, which were smaller (1-2+) (FIGS. 2A & B). These results were similar and indicated that clot lysis by the two activators was comparable.

In the tPA dog with a 4+ clot, a cast of the segment (2B first tPA photo), the clot was non-adherent, whereas residual thrombi were invariably tightly adherent. This suggested it was due to rethrombosis, a conclusion consistent with its negligible radioactivity.

The discrepancy between the radioisotope and anatomic findings with M5 indicated that in the 5 dogs with little (one) or no (four) thrombi, the radioactivity was found to come from the vessel wall and segment bed. Why this diffusion of radioactivity during lysis occurred more with M5 than tPA remains to be explained.

The addition of a flow probe as a substitute was attempted but abandoned because it required frequent adjustments which interfered with keeping the isotope monitor in position.

### Rebleeding From the Four Injury Sites

Rebleeding was defined as blood loss at a wound site >1,000 ODU (˜1.3 ml). Rebleeding with tPA tended to occur at multiple sites per dog, being at 3 or more sites in four dogs, 2 sites in one, and 0 sites in one (mean 2.7 sites). Rebleeding with M5 occurred at 3 sites in only one dog and at 0 or 1 site in the remainder (mean 1.2 sites) (p=0.062) (FIG. 3).

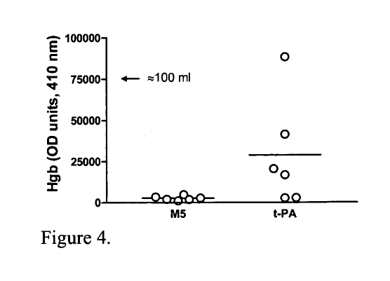

Total blood loss (all sites combined) averaged >30,000 ODU (˜40 ml whole blood) in the tPA dogs compared with <3,000 ODU (˜4 ml) in the M5 dogs (p<0.026). Four of the tPA dogs bled extensively with one almost exsanguinating (>80,000 ODU or >110 ml whole blood) (FIG. 4).

### Blood Analyses From the Dog Samples

At 55 minutes, the fibrinogen concentrations (mean and range), expressed as a percent of the baseline value, were 60% (47-85) for tPA and 82% (58-100) for M5. At 90 minutes they were similar, being 66% (46-100) and 82% (46-98) for tPA and M5 respectively. The differences between the tPA and M5 fibrinogen values were not statistically significant.

Zymography of the 55-minute samples alongside a range of M5 concentrations indicated the mean M5 plasma concentration in the dogs during the infusion to be about 8 μg/ml (data not shown).

### Zymograms of tcM5 and UK Incubated in Plasma

Zymograms of equal concentrations of tcM5 or UK incubated in vitro in either human or dog plasmas showed that both tcM5 and UK appeared as higher (˜45 kDa) and as lower (˜30 kDa) molecular weight bands of activity. The lower band is a more degraded by-product of plasmin-activation of their respective single chain forms routinely seen by this method. In addition, two inhibitor complexes appeared within minutes of incubation of tcM5 in plasma. The position of the predominant inhibitor complex with tcM5 was consistent with C1-inactivator (˜150 kDa) and the lower, less prominent band with an antithrombin complex (˜110 kDa), which is a known inhibitor of UK. These complexes appeared later and were far less prominent with UK. C1-inactivator has not been listed among the known plasma inhibitors of UK (Murano, et al., Blood 55: 430-436 (1980)). A corresponding more rapid loss of the free enzyme activity bands at 45 and 30 kDa was also seen with tcM5 compared with UK. Complexes with PAI-1 were not visible, probably due to the overwhelming concentrations (5 μg/ml) of the activators. Despite the presence of high molecular weight (HMW) and low molecular weight (LMW) forms of the free enzymes, the inhibitor complexes appeared as single bands of activity, indicating non-resolution of the two forms. These findings were reproducible and comparable in human and dog plasma. A representative zymogram from an experiment in human plasma is shown (FIG. 5).

Zymograms from plasma samples obtained in vivo from the six M5 dogs showed only a ˜45 kDa band with no detectable complexes, indicating little systemic tcM5 generation (consistent with the little fibrinogen degradation). However, when samples were examined from higher doses used during dose-finding and in which a significant non-specific effect occurred, inhibitor complexes comparable to those seen in the in vitro studies were seen (data not shown).

### C1-Inactivator:tcM5 Complex Identification (FIG. 6)

Western blotting with antibodies to UK (A, lane 3,4) and C1-inactivator (A, lane 5) of plasma samples containing tcM5 revealed complexes migrating in the same position with each other and with the predominant complex in a zymogram of this plasma (A, lane 1). However, control plasma showed a C1-inactivator complex in a similar position, probably with factor X11a (not shown). Since the two complexes could not be satisfactorily resolved, studies in a purified system with C1-inactivator and tcM5 were done. A zymogram of tcM5 (B, lane 1) and a mixture of tcM5 and C1-inactivator (B, lane 3) revealed that a complex formed which migrated in the same position as that seen on the plasma zymograms. Similarly, Coomassie staining of SDS PAGE of tcM5 (B lane 4), tcM5 plus C1-inactivator (B lane 5), and C1-inactivator (B lane 6) showed the same enzyme:inhibitor complex. The faint band below C1-inactivator in B lane 5 probably represents a tcM5 antithrombin complex. MW markers are shown in lanes A2, B2 and 7 (FIG. 6).

### Discussion

In a previous in vivo study, M5 caused effective lysis of lung clots in dogs with little bleeding from two hemostatic sites, suggesting that hemostatic fibrin was spared by M5 (Liu et al., Circ Res. 90: 757-763 (2002)). Due to the unusual nature of this effect and its potential clinical application, a critical reexamination was conducted with the following protocol modifications: an arterial thrombus was substituted, more injury sites were created, blood loss was precisely quantified by a novel technique, M5 dosage was reduced, and tcM5 inhibition in plasma was studied.

Examination of the femoral artery segments at 90 minutes (30 minutes post-infusion) showed effective lysis by M5 with small (1-2+) adherent residual thrombi in only two out of six dogs. In the tPA dogs, there were thrombi (3-4+) in three dogs. However, one of these (4+ thrombus) was due to rethrombosis, based on its non-adherence to the vessel wall and negligible radioactivity. Therefore, the efficacy of thrombolysis by M5 and tPA was comparable. Although in the previous dog study clot lysis by M5 was superior to tPA, the M5 dose in that study was 40% higher and the tPA dose 40% lower (Liu et al., Circ Res. 90: 757-763 (2002)).

The radioisotope counts over the segment suggested that lysis was slower and less complete with M5. The discrepancy between this surrogate endpoint and the above findings indicated that in the five M5 animals with little or no residual thrombus (FIG. 2B), the radioactivity came from elsewhere, and it was found to emanate from the vessel wall and surrounding tissue. Why this occurred more with M5 than tPA remains to be determined. In addition, due to rethrombosis in one tPA dog, the radioisotope findings were not a reliable indicator of the vessel lumen content for either activator in this study.

Due to the small size of this study, little significance can be attached to the finding of rethrombosis in one of the tPA animals. However, this event is consistent with previous studies in tPA-treated dogs (Rapold et al., Blood 77: 1020-1024 (1991)), with clinical studies in which a 25-30% early coronary reocclusion incidence was reported with tPA (Wilson et al., Am Heart J. 141: 704-710 (2001)), and with reports that the efficacy of percutaneous coronary intervention over tPA was related to a higher reocclusion rate with tPA (Armstrong et al., Circulation 107: 2533-2537 (2003)). By contrast, coronary reocclusion rates of only 0-5% were reported with proUK (Pannell and Gurewich, Blood, 67: 1215-1223 (1986), Weaver et al., J Am Coll Cardiol. 24: 1242-1248 (1994), Zarich et al., J Am Coll Cardiol. 26: 374-379 (1995)), and markers of thrombin generation in plasma were not induced by proUK (Weaver et al., J Am Coll Cardiol. 24: 1242-1248 (1994)), in contrast to tPA (Owen et al., Blood. 72: 616-620 (1988)).

In conclusion, M5 is a single site mutant of proUK with a lower intrinsic activity and thereby superior stability in plasma, which enables its pro-enzyme fibrinolytic properties to be better preserved at therapeutic concentrations. In dogs, M5 and tPA induced lysis of a femoral artery thrombus with comparable efficacies, but M5 caused ten-fold less (p=0.026) bleeding from wound sites due to an apparent sparing of hemostatic fibrin. This was postulated to be due to the finding that plasminogen activation by M5 was not promoted by intact (hemostatic) fibrin, and to the efficient inhibition of tcM5 by plasma C1-inactivator.

1. A method of reducing bleeding during fibrinolysis treatment in a patient, the method comprising:
(a) providing a pro-urokinase mutant polypeptide (M5) wherein the amino acid Lysine has been replaced by the amino acid Histidine at position 300;
(b) administering an amount of the M5 effective to cause the lysis of an occlusive blood clot; and
(c) administering an amount of exogenous C1-inactivator sufficient to establish concentrations greater thah physiological levels in the plasma of the patient.

2. (canceled)

3. The method of claim 1 wherein the C1-inactivator is administered prior to the infusion of M5 in amounts sufficient to establish concentrations of at least about 0.75 g/l in the plasma of the patient.

4. The method of claim 1 wherein M5 is mixed with a pharmaceutically acceptable carrier and administered as a bolus of about 20 to 60 mg.

5. The method of claim 1 wherein M5 is mixed with a pharmaceutically acceptable carrier and administered by intravenous infusion at a rate of about 40 to 80 mg/hour.

6. The method of claim 1 wherein M5 is mixed with a pharmaceutically acceptable carrier and administered by intravenous infusion at a rate of up to 200 mg/hour.

7. A method of inhibiting the enzymatic activity of the two-chain urokinase mutant tcM5 comprising administering to a patient an amount of exogenous C1-inactivator sufficient to limit the formation of tcM5 from M5 activation, wherein M5 is a pro-urokinase mutant polypeptide in which the amino acid Lysine has been replaced by Histidine at position 300.

8. The method of claim 7 wherein the amount of C1-inactivator administered is sufficient to establish concentrations substantially greater than physiological levels in the plasma of the patient.

9. The method of claim 8 wherein the amount of C1-inactivator administered is sufficient to establish concentrations of at least about 0.75 g/l in the plasma of the patient.
